(19) United States
(12) Plant Patent Application Publication
(10) Pub. No.: US 2003/0188361 P1
(43) Pub. Date: Oct. 2, 2003

(54) Ornamental sweetpotato plant named 'Sweet Caroline Purple'

(76) Inventors: Kenneth Pecota, Raleigh, NC (US); G. Craig Yencho, Washington, NC (US); Cynthia Pierce, Clayton, NC (US)

Correspondence Address: MYERS BIGEL SIBLEY & SAJOVEC, PO BOX 37428, RALEIGH, NC, 27627, US

(21) Appl. No.: 10/114,502

(22) Filed: Apr. 2, 2002

(51) Int. Cl. A01H 5/00

(52) U.S. Cl. PLT/258

(57) ABSTRACT

A new and distinct ornamental cultivar of Ipomoea batatas called ‘Sweet Caroline Purple’ is described that is a moderately compact, densely-mounding, plant with purple leaves that are deeply-lobed. The plant is distinguished by its short shoots and highly branched plant habit. Furthermore, this plant has excellent vigor and will flower under short day conditions. Ipomoea batatas ‘Sweet Caroline Purple’ is suitable for use in landscaping and containerized gardens.

### LATIN NAME OF THE GENUS AND SPECIES

The Latin name of the novel, ornamental plant variety disclosed herein is Ipomoea batatas (L.) Lam.

### VARIETY DENOMINATION

The inventive cultivar of Ipomoea batatas disclosed herein has been given the variety denomination ‘Sweet Caroline Purple’.

### BACKGROUND OF THE INVENTION

Ipomoea batatas species are members of the morning glory family Convolvulaceae. Ipomoea batatas, commonly referred to as the white or yellow sweetpotato and the orange yam, are typically fast growing vines with palmately-lobed leaves. These ornamental species produce storage roots identical in appearance to the common sweet potato, but not as palatable. Late in the growing season, tubular flowers appear which are similar to morning glories, but plantings are dominated by the appearance of the foliage. The plants are highly desirable due to their ability to grow under varied stress conditions, cover a large space, and last the entire growing season. Moreover, these plants have few insect or disease problems.

Existing varieties of Ipomoea batatas are popular for landscaping applications. There are currently six common types of ornamental sweetpotatoes that are being cultivated primarily for annual, summer vines. These six cultivars are: ‘Blackie’, having dark purple-black foliage, lavender flowers, and edible storage roots; ‘Terrace Lime’ and ‘Margarita’ (also known as ‘Sulfur’), which have large brilliant chartreuse leaves and lavender blooms; ‘Black Heart’ (also known as ‘Ace of Spades’), having heart-shaped leaves with burgundy purple color; ‘Tricolor’ (also known as ‘Pink Frost’), is a variegated plant which has pale green, white, and pink-margined leaves; and ‘Lady Fingers’, which has medium green, dainty leaves divided into long, thin, fingerlike lobes which are complemented by burgundy stems and veins.

Ipomoea batatas ‘Margarita’ has recently been released in the United States, and has become widely used as a landscape annual. It is not suitable for mixed containers, as this variety exhibits a very vigorous growth and tends to out-compete other species. Another popular variety is ‘Blackie’, a vigorous purple-leaved clone which is also unsuited to containerized gardens. See Armitage. A. M. and J. M. Garner. 2001. Ipomoea batatas ‘Margarita’. HortScience 36:178.

Therefore, to meet the current horticultural demand, it is desirable to produce new, more robust cultivars of ornamental sweetpotato with attractive foliage colors, leaf shapes, and plant architectures. In addition, it would be advantageous to develop cultivars of ornamental sweetpotato exhibiting a more compact growth, and which do not out-compete other species in mixed containers.

The present invention comprises a new and distinct variety of Ipomoea batatas, which has been named ‘Sweet Caroline Purple’. The variety is suitable for use as a landscape or containerized plant.

Lineage.

The Ipomoea batatas ‘Sweet Caroline Purple’ cultivar originated from a conventional cross between Ipomoea batatas cultivars Sulfur (the female parent; not patented) and Blackie (the male parent; not patented) conducted in the Winter of 1997-1998 at the Horticultural Greenhouses located at North Carolina State University, Raleigh, N.C. Seeds from this cross were planted in the Horticultural Greenhouses in Spring 1998 and in a field at North Carolina State University in June 1998. The single, individual plant now known as Ipomoea batatas ‘Sweet Caroline Purple’ was selected in October, 1998 because of its combination of exceptional features, and has been propagated asexually since that time.

Asexual Reproduction.

Since its selection, Ipomoea batatas ‘Sweet Caroline Purple’ has been asexually reproduced at the Horticultural Greenhouses located at North Carolina State University, Raleigh, N.C. by vegetative propagation of vine cuttings. Successively, there have been four cycles of vegetative propagation, one cycle of tissue culture micropropagation, followed by multiple vegetative propagation cycles to increase the plant population. The combination of characteristics disclosed herein for Ipomoea batatas ‘Sweet Caroline Purple’ has remained stable through the successive generations of asexual reproduction.

### BRIEF SUMMARY OF THE INVENTION

The present Ipomoea batatas ‘Sweet Caroline Purple’ ornamental plant is a compact, densely-mounding cultivar producing many, short shoots. This cultivar is distinguishable from other cultivars by its purple leaves that have 3-5 deep lobes and a compact, highly branched plant habit. Furthermore, ‘Sweet Caroline Purple’ has a good vigor and will flower under short day conditions.

### BRIEF DESCRIPTION OF THE DRAWINGS

FIG. 1 is a color photograph of a typical specimen of Ipomoea batatas ‘Sweet Caroline Purple’ (plant on right) compared with the commercially available ‘Blackie’ cultivar (plant on left).

FIG. 2 shows the variety of leaves produced by Ipomoea batatas ‘Sweet Caroline Purple’ and the lower surface of the leaf (bottom row, center leaf).

### DETAILED BOTANICAL DESCRIPTION

The following is a detailed description of the botanical characteristics of a new and distinct cultivar of Ipomoea batatas plant known by the cultivar name Ipomoea batatas ‘Sweet Caroline Purple’. All colors cited herein refer to The Royal Horticultural Society Colour Chart designations except where general terms of ordinary dictionary significance are used. Where dimensions, sizes, colors, and other characteristics are given, it is to be understood that such characteristics are approximations or averages set forth as accurately as practicable.

The descriptions reported herein are from ten-month-old specimens grown individually in ten-inch azalea pots. The plants were grown in Raleigh, N.C., under commercial practice in a glass-covered greenhouse where, during the winter, day and night temperatures range between 18-30° C. and 15-21° C., respectively. Plant histories were taken in March 2002. Ipomoea batatas ‘Sweet Caroline Purple’ has not been observed under all possible environmental conditions; therefore, the phenotype may vary with variations in the environment such as season, temperature, light intensity, day length, cultural conditions, and the like.

Growth Conditions.

Ipomoea batatas ‘Sweet Caroline Purple’ has excellent vigor, has a moderately fast growth rate, and is very adaptable to container culture. In locales with mild winter conditions, Ipomoea batatas ‘Sweet Caroline Purple’ will grow perennially; otherwise it is an annual plant. Similar to cultivated sweetpotatoes, wind or rain rarely causes much damage to ‘Sweet Caroline Purple’, but if damage does occur, the plant drops the damaged leaves and grows new shoots at nodes where the leaves were lost. Under low light levels in a greenhouse, ‘Sweet Caroline Purple’ can develop intumescence, which will remain on the affected foliage, but will be outgrown with new foliage.

Above-Ground Structure and Coloration.

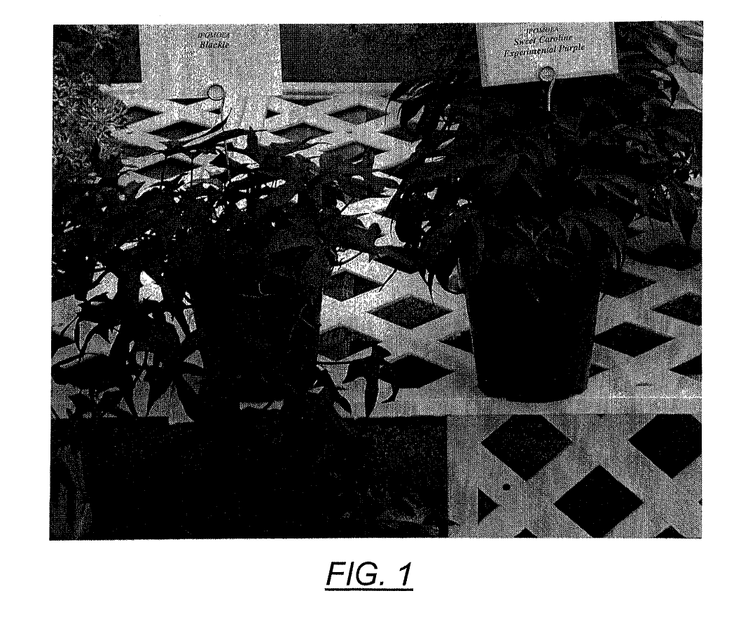

FIG. 1 shows the shape and coloration of a typical specimen of Ipomoea batatas ‘Sweet Caroline Purple’. Color will vary somewhat due to temperature and nutrient stress, which affect the anthocyanin pigments (which give rise to the purple color). Overall, this cultivar is a compact, round-mounded, herbaceous plant that has an average height of 18 cm and an average area of spread of 108 cm. The growth habit of this plant is to grow upright with shoots growing outward.

Branching Habitat.

Freely-branching with ˜14 lateral branches coming off the stem. Very dense foliage with no pinching required to stimulate branching.

Vegetative Lateral Branches.

Length: ˜35 cm. Diameter: ˜0.3 cm. Internodes are short with an average length of ˜1.5 cm. Many lateral branches are formed and each axil has latent shoots.

Stem.

Round and smooth with an outward and upward bending aspect and very good strength. Color: 187A.

Petiole.

Length: ˜8.4 cm, Diameter: ˜0.2 cm. Color: 187A.

Foliage.

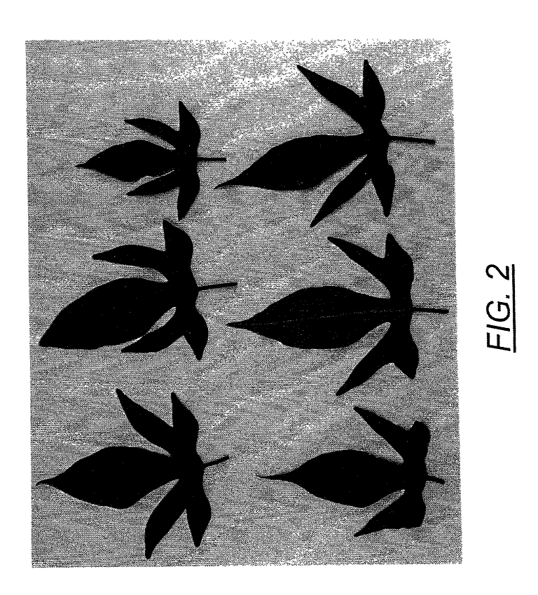

Leaves are alternate and simple, but deeply divided into 3-5 lobes. Leaf shape is somewhat variable as is size (see FIG. 2). Quantity: Densely foliated, with ˜16-18 leaves per lateral branch. Mature leaf length: ˜9.3 cm. Mature leaf width: ˜9.2 cm. Leaf margin is entire. Lobe width: ˜1.4 cm. Mid-vein lobe length: ˜7.9 cm. Mid-vein lobe width: ˜2.9 cm. Leaf apex: Acute. Leaf base: Acute. Leaf has a smooth texture and matte finish. Venation is palmate at the base with arcuate veins in the center lamina. Color: Table 1. Leaves go from green to purple as they mature.

TABLE 1

| Leaf Structure | Upper Surface | Lower Surface |
|---|---|---|
| Young Leaf | 144A | 144A |
| Mature Leaf | 187A | 187A |
| Vein-mature leaf | 187A | 79A |
| Vein-young leaf | 144A | 144B |

Flowers.

Flowers occur under short day conditions, but the precise photoperiod for flower induction is currently unknown. The inflorescence is generally a cyme in which the peduncle is divided into two axillary peduncles. Each peduncle is further divided into two after the flower is produced. Usually buds of the first, second and third order are developed, but sometimes single flowers are produced. Corolla width: ˜4.0 cm, corolla length: ˜3.9 cm. Limb color: 69D. The throat color gets lighter from base to limb going from 78A at the base to 78D near the limb. The limb is pentagonal. The two outer sepals are shorter than the inner sepals. The sepals are elliptic and purple in color. The stigma and style are both white. The stigma is exerted relative to the stamens. Descriptions are based on: CIP, AVRDC, IBPGR. 1991. Descriptors for Sweet Potato. Huaman, Z., editor. International Board for Plant Genetic Resources, Rome, Italy.

Storage Root Coloration.

The storage roots have a white skin with cream-colored flesh.

Comparison with Other Ipomoea batatas Cultivars.

Of the six most common cultivars of ornamental sweetpotato, Ipomoea batatas ‘Sweet Caroline Purple’ most resembles ‘Blackie’. Like ‘Blackie’, Ipomoea batatas ‘Sweet Caroline Purple’ has purple, deeply lobed leaves. However, ‘Sweet Caroline Purple’ has a moderately-compact plant habit compared with the trailing habit of ‘Blackie’. Furthermore, ‘Sweet Caroline Purple’ is highly branched compared with ‘Blackie’ resulting in a much denser foliage appearance. As compared with ‘Blackie’, ‘Sweet Caroline Purple’ has a more bunched and fuller appearance, which is more suitable for containerized propagation.

In a comparison with the parental strains, ‘Sweet Caroline Purple’ shares some attributes with its paternal parent (Table 2), but is also quite distinct therefrom and comprises a unique combination of characteristics.

TABLE 2

| | ‘Sweet Caroline | Female Parent | Male Parent |
|---|---|---|---|
| Characteristic | Purple’ | ‘Sulfur’ | ‘Blackie’ |
| Plant Habit | Moderately Compact | Trailing | Trailing |
| Foliage Color | Purple | Light Green | Purple |
| Leaf Size | Moderate | Large | Moderate |
| Leaf Shape | Deeply Lobed | Slightly Lobed | Deeply Lobed |

Disease or Pest Resistance.

‘Sweet Caroline Purple’ is susceptible to Sweetpotato Feathery Mottle Virus and damage by Japanese beetles.

Herbarium Vouchers.

A voucher of ‘Sweet Caroline Purple’ was obtained by Dr. Paul R. Fantz and Dr. Robert Lyons, Department of Horticultural Science, North Carolina State University, Raleigh, N.C., on Jul. 6, 2001. The voucher number is 6576, and the voucher will eventually be deposited into the Herbarium of North Carolina State University (NCSC) in Raleigh, N.C.

What is claimed is:

1. A new and distinct cultivar of Ipomoea batatas plant named ‘Sweet Caroline Purple’, substantially as illustrated and described herein.
